(19) United States
(12) Patent Application Publication
(10) Pub. No.: US 2004/0243021 A1
(43) Pub. Date: Dec. 2, 2004

(54) Device for thermal stimulation of small neural fibers

(76) Inventors: John C. Murphy, Clarksville, MD (US); Rafal P Szczepanowski, Hanover, MD (US); Wolfger Schneider, Columbia, MD (US); Richard A Meyer, Columbia, MD (US); Justin C McArthur, Phoenix, MD (US); Michael Polydefkis, Baltimore, MD (US)

Correspondence Address: Francis A. Cooch Office of Patent Counsel, The Johns Hopkins University, Applied Physics Laboratory, 11100Johns Hopkins Road, Laurel, MD, 20723-6099, US

(21) Appl. No.: 10/494,584

(22) PCT Filed: Nov. 5, 2002
(86) PCT No.: PCT/US02/35525

Related U.S. Application Data
(60) Provisional application No. 60/332,785, filed on Nov. 6, 2001.

(51) Int. Cl. A61B 5/00

(52) U.S. Cl. 600/549; 600/555

(57) ABSTRACT

A device operable to assess temperature response of small neural fibers. The device includes a heat source. A skin contacting probe tip that includes at least one skin contacting region is operatively connected to the heat source and operable to apply a heat to regions of skin having varying surface areas. A temperature sensor is arranged in the vicinity of the probe tip and is operable to detect a temperature of the probe tip. A controller is operatively connected to the heat source and the temperature sensor to maintain a target temperature of the probe tip.

### REFERENCE TO RELATED APPLICATIONS

This application claims the benefit of prior filed co-pending U.S. provisional application No. 60/332,785, filed on Nov. 6, 2001.

### FIELD OF THE INVENTION

The present invention relates to a diagnostic tool and method for testing patient skin response to elevated temperature.

### BACKGROUND OF THE INVENTION

There is need to determine the distribution of peripheral nerve response on the body for a variety of medical conditions including those which cause loss of response from disease or injury and for management of acute pain caused by a variety of conditions. A perception of pain is produced by near simultaneous activation of an ensemble of sensors in the skin each kind individually responsive to a specific stimuli e.g. pressure, needle stick, heat. A pain perception occurs when the number of sensors activated produces a synaptic response. A result is that pain perception involves three parameters: the presence of a stimulus specific to the nerve fibers of interest; the response of individual nerve fibers whose threshold for response is below the value of the stimulation; the number of nerve fibers activated which if the area of activation is known is directly related to nerve fiber density. No convenient thermal stimulation device is currently available.

### SUMMARY OF THE INVENTION

The present invention provides a device operable to assess temperature response of small neural fibers. The device includes a heat source. A skin contacting probe tip is operatively connected to the heat source and is operable to apply heat to regions of skin having varying surface areas. A temperature sensor is arranged in the vicinity of the probe tip and is operable to detect a temperature of the probe tip. A controller is operatively connected to the heat source and the temperature sensor to maintain a target temperature of the probe tip.

The present invention also includes a device operable to assess temperature response of small neural fibers. The device includes a gripable housing. A heat source is arranged in the housing. An interchangeable skin contacting probe tip extends from an end of the housing. The probe tip is operatively connected to the heat source and is operable to apply non-injurious heat to regions of skin having varying surface areas. A temperature sensor is arranged in the vicinity of the probe tip and is operable to detect temperature of the probe tip. A controller is operatively connected to the heat source and the temperature sensor to maintain a target temperature of the probe tip. A retractable probe tip shield extends around at least a portion of the probe tip when the probe tip is not in use.

Additionally, the present invention includes a method for testing patient response to excitation of neural fibers. The method includes contacting a region of a patient's skin with a probe tip that is operatively connected to a heat source. The probe tip is operable to apply non-injurious heat to the region of the patient's skin. Heat is applied to the probe tip with the heat source. A temperature of the probe tip is sensed with a temperature sensor. The temperature of the probe tip is controlled to maintain a target temperature. Patient response to the contact with the probe tip is monitored. At least one of the temperature of the probe tip, a size of the region of the patient's skin that the probe tip contacts, and a shape of the region of the patient's skin that the probe tip contacts is varied.

### BRIEF DESCRIPTION OF THE DRAWINGS

Objects and advantages of the present invention will be more clearly understood from the following specification when considered in conjunction with the accompanying drawings, in which:

FIG. 1 represents an overhead view of an embodiment of a device according to the present invention;

FIG. 2 represents an electrical schematic drawing of a circuit diagram for an embodiment of a device according to the present invention;

FIG. 3 represents a diagram of another embodiment of a device according to the present invention;

FIG. 4 represents a side view of a portion of an embodiment of a device according to the present invention;

FIG. 5 represents a side view of another portion of the embodiment shown in FIG. 4;

FIG. 6 represents a perspective view of another portion of the embodiment shown in FIGS. 4 and 5;

FIGS. 7 and 8 represent cross-sectional views of a portion of the embodiment shown in FIGS. 4, 5, and 6; and

FIG. 9 represents a perspective view of the embodiment shown in FIGS. 4, 5, 6, and 7.

### DETAILED DESCRIPTION OF THE INVENTION

The present invention at least in part concerns a sensor for thermal pain produced by a class of neural fibers exhibiting a threshold response to temperature near 45° C. in healthy individuals. Measurement of the threshold response temperature and its distribution over the body is one objective of this invention. In addition, since stimulation of single nerve fibers does not produce a pain response it is desirable to determine the number and/or the density of nerve fibers which when activated above threshold produce a response.

Another specific goal of this invention is to produce a device to determine both threshold response and neural fiber density at various locations on the body in a rapid and convenient manner. Such thermal pain sensing could be important in mapping neuropathy associated with disease and has potential advantages over other methods of stimulating a pain response such as pricking with a sharp needle.

Concerning the potential advantage of thermal versus mechanical pain stimulation, the stimulus area, and volume, when depth of stimulation is considered, produced by a thermal probe is expected to be approximately equal to the area of the probe in contact with the skin since the thermal diffusion length both laterally and in depth is smaller than the smallest planned probe sizes. The thermal diffusion length≈[k t₀]^{1/2} where the thermal diffusivity of skin k≈0.0015 cm² sec⁻¹ and t₀ is the duration of heating. For heating times of 10 seconds, the thermal diffusion length is approximately 1 mm, which is substantially smaller than the smallest probe diameter being considered. The diffusion length also sets 1 mm as the approximate depth of heating in the tissue.

Conversely, needle pain stimulation can deform the skin over distances of several mm's and produces a response with depth that is limited primarily by the needles ability to penetrate the skin without deformation. This makes the process dependent on the variation in skin mechanical properties and likely produces pain spread over a wider area and at greater depth and hence larger volume than for thermal activation. This limits the ability to make reliable measurements of neural fiber density.

It is expected that thermal stimulation will be valuable in determining changes in small fiber response to temperature with time and location on the body. By varying the heated area in contact with the skin it should be possible to determine the density of neural fibers with good accuracy. The combination of threshold temperature response and nerve fiber density then provides a basis for monitoring disease onset and progression and evaluating the efficacy of methods of reducing pain. Furthermore, the probe may be of value in a research seeking to determine the variation in response in the population at large.

A computer based system according to the present invention can permit integrated management of patient data and even automation of the entire evaluation process.

The present invention provides a device that can thermally stimulate regions of a patient's skin. As such, the present invention can thermally stimulate small neural fibers. Through such thermal stimulation, the present invention can help determine pain response elicited by excitation of small nerve fibers. The present invention also easily permits the size, shape, and/or pattern of the region of a patient's skin producing thermal pain to be identified and mapped.

The present invention permits control over the thermal stimulation so that a threshold response to temperature at various locations on the body may be determined. Nerve fibers that have a threshold response do not produce a response until a critical temperature is reached over a minimal area of a patient's skin linked to the density of neural fibers present. Once a critical combination of temperature and surface area of stimulation are reached, the patient perceives pain. The pain perception can be monitored through responses produced by the patient, such as a verbal report of pain.

A device according to the present invention includes a skin-contacting probe tip. The surface area, shape and pattern of the skin-contacting surface(s) may vary. For example, the tip skin contact surface could have a circular form. The area of the circle could vary in diameter as discussed below. The skin-contacting surface could also have other forms such as a series of parallel rectangular ridges of equal width. Such a skin-contacting surface could permit the directionality of the neural response to be probed. The total contact area could be held constant by controlling the product of the number of ridges and their width. Without changing the contact area, the directionality and spacing of the stimulus would be varied.

Alternatively, the contact surface could have the form of a chessboard with raised black squares. The edge length of the form could be varied. Again, the total contact area may be held fixed as the edge length varies. Hence, the density and texture of the response could be varied.

In general, varying the surface area can permit detection of a density threshold, as described above, but other factors such as directionality and density might be used to evaluate other aspects of the neural response. Altering any aspect of the skin contacting surface may be carried out by replaceable tips as described herein or through having a skin contacting surface that can be varied.

A skin contacting surface area may be altered by providing the device with interchangeable tips. A device that includes interchangeable tips may include a mechanism on the tips and/or on other portions of the device to permit the tips to be changed. Typically, the interchangeable tips include a mechanism between the tip and the body of the device to permit rapid tip interchange. For example, the tips could screw into a base, or the tips and/or the base could include protrusions and/or detents. Additionally, spring and leaf capture elements may be employed. Any suitable interconnecting means could be utilized.

Rather than including interchangeable probe tips, the probe tip could be expandable and contractable to vary the area of the skin-contacting surface. For example, the probe tip could be fashioned similar to a camera diaphragm. Alternatively, movable members could be included that could move into and out of position to make the skin contacting surface bigger or smaller.

Similar means could also be employed to alter the pattern of the skin contacting surface. For example, interchangeable probe tips could include skin-contacting surfaces having different patterns. Alternatively, different portions of a probe tip could be moved to alter the skin contacting surface.

The material used for the probe tip may also vary. Typically, the tip is made of a material that is a good heat conductor allowing rapid transfer of heat from the probe to the skin and minimizes temperature differences over the tip. The temperature may be actively controlled by feedback from a temperature sensor in the tip. In this case, the temperature sensor typically is located as closely as possible to the tip-skin interface and the tip material typically is has a high thermal effusivity.

According to one embodiment, the probe tip is made of gold flashed aluminum. Since aluminum alloys typically vary in thermal conductivity by a factor of about three, optimal alloys may be selected for best performance. Similar considerations may apply to other materials discussed below. Other materials that the tip could be made of include other metals, such as copper, gold and silver and some non-metallic materials, such as silicon or sapphire. Typically, the material that the probe tip is made of has good heat transfer properties.

According to one embodiment, the tip is formed as a hollow cylinder. The temperature sensor is inserted in the hollow cylinder with its active area close to the bottom of the hollowed region close to tip-skin interface. This arrangement can allow good control of the tip temperature both in temperature accuracy under stationary conditions and in maintenance of temperature with time after the tip is placed in contact with the skin. Small changes in temperature associated with heat transfer from tip to skin may be rapidly compensated by the active control system provided the tip surface and probe are in good thermal contact.

According to one embodiment, the device includes interchangeable skin contact probes that include a truncated cone having a cylindrical skin contact surface. The skin contact area has a diameter of about 2 mm to about 12 mm. The probe tip may be made of thermally conductive materials such as aluminum, copper, silver, gold and the other materials discussed above.

In yet another embodiment, the probe tip material is made of a thin crystal silicon wafer bonded to one end of a small hollow polymer cylinder metallized on its interior surface. An optical fiber would couple light energy from a LED source in the housing into the open end of the polymer cylinder. The silicon is anodized to increase light absorption and reduce reflectance. As a result, entering light from the fiber is both absorbed and reflected from the silicon surface and also reflected from the walls of the polymer creating a small quasi-black body cavity. A temperature probe can be mounted in the wall of the tube near the silicon wafer end to monitor temperature for closed loop control at minimal temperature error. Alternatively, the end of the optical fiber could contain thermally sensitive fluorophores which would be directly excited by the radiant energy from the LED and produce fluorescence that would return up the optical fiber to the housing for detection. This configuration would then combine optical heating and optical detection of local temperature at the tip. As a final detection temperature measurement concept, the silicon wafer itself, if appropriately doped could be used to measure its own temperature through changes in conductivity. This would remove any ambiguity in temperature reading associated with heat transfer impedance between tip and temperature detector.

The probe tip is heated with a heat source. Typically, the heat source is in good thermal contact with the tip permitting rapid transfer of heat to the skin-tip interface so that the bandwidth of the temperature feedback loop controlling the tip surface temperature can be wide enough to permit rapid alteration and control of tip temperature. The heat source typically also permits precise temperature control, has a low thermal mass, good thermal conductivity and a low specific heat. The heat source can be a resistance heating element. Alternately, it could be an optical source with light energy delivered through an optical fiber. One resistance heating embodiment includes a resistively heated polymer film that includes a plurality of small diameter resistance wires. Other embodiments include a continuous polymer film containing conductive particles at specified concentrations or utilize optical heating using a Light Emitting Diode in a housing with a fiber optic cable used to deliver light to the tip. The optical design may offer some advantages in tip design and exchange, cost and minimize unwanted heat leakage.

The combination of the heat source, the closed loop control system containing the source, the temperature detector, the feedback electronics and close integration of the temperature detector in the tip can collectively determine temperature accuracy and any time dependent temperature error. These elements can provide precise temperature control. The use of proportional-integral control system in the control electronics can provide low stationary temperature offset errors and low time response errors.

The temperature of the probe tip may vary over a range from the normal physiological temperature of the free skin surface, which is about 32° C., to a temperature selected to be well above the thermal pain threshold for diseased skin, which may be about 53° C. Different upper and lower temperature values can be chosen depending upon circumstances of use but may always be chosen to be non-injurious. The highest temperatures chosen typically will not burn, blister or otherwise damage a patient on which the device is being utilized.

To help maintain control of the temperature of the probe tip, a device according to the present invention includes a temperature sensor. The temperature sensor may be arranged to sense the temperature of the skin-contacting surface of the probe tip. Typically, to sense the temperature of the probe tip accurately, the sensor should not experience direct thermal leakage from the heat source or other regions of the probe. Such isolation of the sensor from the heat source may be accomplished through placement of the sensor relative to the heat source and/or thermal insulation of the sensor from the heat source.

Many types of sensor can effectively monitor the temperature of the probe tip. A sensor with good temperature sensitivity and accuracy over the range of desired control temperatures and a response time much less than the thermal diffusion time in tissue is desirable. These characteristics are particularly desirable to maintain the programmed temperature chosen for patient testing. Some examples of sensors include surface mount thermistors, thermocouples, semiconducting diodes and semiconductors, such as silicon, whose resistance varies with temperature. Another class of materials includes selected fluorescent glasses and ceramics whose fluorescence intensity varies with temperature.

The heat source may be connected to the probe tip in a manner that effectively permits heat to be transferred to the probe tip. The sensor and the heat source typically are arranged such that the sensor does not partially or completely sense the temperature of the heat source directly as opposed to the temperature of the probe tip. If the sensor senses temperature of the heat source directly this can produce a sensor temperature somewhat higher than the tip temperature and hence the skin-contacting surface temperature would be lower than the temperature set by the feedback loop. Thermal insulation can help to reduce unintended heat leaks and greatly improve temperature accuracy and response time. Optical heating discussed above may be an especially attractive method for providing heat to the probe surface with minimum heat leak to the sensor and even greater patient safety.

The device may be controlled with one or more controllers operatively connected at least to the heat source and the sensor. The controller(s) may be included in a hand held portion of the device. Alternatively, the controller(s) may be included in a separate device, such as a desktop, laptop, handheld or other computer that the device of the invention is operatively connected to. The connection between the hand-held portion of the device and the controller may be wired or wireless. A wireless connection could include a radio frequency, such as Bluetooth or other protocol, or infrared connection. A wired or wireless connection may be employed for selection and recording functions and permit remote data collection and analysis.

The controller(s), the heat source, and the temperature sensor can be operatively connected to form a feedback circuit. The circuit can drive the probe tip to a target temperature and maintain the temperature. Many types of controller may be utilized with many types of control system. Typically, the controller(s) should have good resolution to avoid excessive control point hunting.

According to one embodiment, the controller maintains the target temperature utilizing both proportional and integral feedback. One specific embodiment employed a personal computer as a controller with a 16 bit A/D card for control of the device. Another embodiment utilizes a five volt microcontroller with ten bit capability. In this embodiment, without amplification, about seven to about ten bits of sensor information were available per degree Celsius for control calculations. One embodiment has a resolution of better than about 0.1 degree Celsius.

The heat source, the sensor, and/or the controller may be operatively connected to a power source. The power source may be arranged in the housing. In such an embodiment, the power source may include one or more batteries or other compact power supply. Alternatively, the power source may be arranged outside of the housing with electrical connection provided by one or more power cables. Along such cables, the device may be connected to a power outlet or to a personal computer and the power supplied with a wired connection. Any transformer or other electrical elements may also be arranged within or outside of a hand held portion.

According to one embodiment, the device is powered directly by a 60 Hz power supply arranged in a hand held portion. Typically, to enhance clinical use, the device is powered by one or more batteries. The batteries may be rechargeable. For example, three 3.6 V nickel metal hydride cells could be employed. Other embodiments may be powered by connecting it to an outlet connected to the power grid. In the U.S., this would include a 110 V AC power supply.

To help in testing, diagnosing and treating patients, the device may include a memory structure. The memory structure may be operatively connected to the device and typically to the controller. The memory could be arranged in a hand held portion. Alternatively, the memory could be arranged in a personal computer, such as a desktop or portable computer, or handheld computer to which the device is attached. If the memory were housed in a hand-held portion, the contents of the memory could be downloaded to one or more computers for analysis. If the device is attached to a hand-held computer, the data could be transferred to a desktop or laptop computer.

Including a memory structure can make it possible to include any parameter related to the operation and use of the device. Along these lines, the data could include test location(s) on patients' bodies, threshold temperature, surface area of skin contact, pattern of skin contact, time period of skin contact with the probe tip, date, time and patient information concerning health status, among others. The data can be compiled, compared, analyzed and manipulated in a number of ways to determine things about an individual patient as well as a population of two or more patients.

As referred to above, a device according to the present invention may include a hand-held portion. The hand-held portion may include a gripable portion. The hand-held portion may house a number of elements of a device. Typically, the hand-held portion may house the probe tip, the temperature sensor, and the heat source. The controller(s), power supply and/or memory could also be housed within the hand-held portion. The hand-held portion may include one or more grip portions to facilitate its retention by an operator.

To facilitate proper heat transfer to a patient and also safety and control of the operation of the device, the device may include one or more shield elements that extend at least partially about the probe tip. Along these lines, the shield could include a plurality of elements that each extend partially about the probe tip. The shield elements could be equidistantly spaced about the probe tip. Alternatively, the shield could include one element that extends completely about the probe tip. The shield may extend at least partially about the probe tip when the device is in use and/or when the device is not in use. The shield can include an insulated housing to help ensure proper heat transfer and maintain temperature of the probe tip.

If the shield extends about the probe tip when the device is not in use, it may extend beyond the end of the probe tip so as to prevent contact with the probe tip. In such an embodiment, the shield may include one or more elements. Also, the element(s) are typically retractable so as to permit the probe to be exposed and make contact with a patient's skin. To help urge the shield into an extended position when the device is not being used to actually contact a patient's skin, as well as to permit interchangeable probe tips to be removed and attached, the device may include one or more springs or other resilient or elastic elements. The elements could urge the shield into an extended position and force applied to the shield could retract the shield against the force of the elements. The spring(s) and/or other elements could be calibrated to help ensure that a certain level of force needs to be applied to retract the shield. Alternatively, the shield could include one or more elements, such as detents or protrusions, that cold lock the shield in an extended position. An operator of the device could manually retract the shield, such as by applying force to the shield.

A device according to the present invention may also include input controls for tuning the device on and off, setting a temperature of the probe tip, as well as other functions. Such functions could also be controlled by or input to a computer, whether desktop, laptop or handheld, that the device is operatively connected to, such as with a wired or wireless connection. Controls for the device could be provided on a hand-held portion described above.

The device may also include elements for indicating the function of the device. Along these lines, the device could include a power indicator and a temperature indicator. The temperature indicator could display the probe tip temperature, the target temperature, and/or whether the probe tip has reached a target temperature. One particular embodiment includes three light emitting elements: a green light emitting element indicates that the device is on and attempting to heat the probe; a yellow light emitting element indicates that the temperature is within a degree band of the target temperature and a red light emitting element indicates that the target temperature has been achieved. Of course, any light emitting elements, such as LED's, light bulbs, or others, could be utilized. Similarly, a text-displaying element could be employed in addition or alternatively. In addition to being operatively connected to the probe tip so as to be able to indicate temperature of the probe tip, the indicator(s) may be connected to a shield as described above. If the indicator(s) is operatively connected to the sleeve, the indicator(s) could indicate a retracted and/or extended position of the sleeve. By indicating a retracted position, the indicator(s) could indicate that the probe tip has contacted a patient's skin.

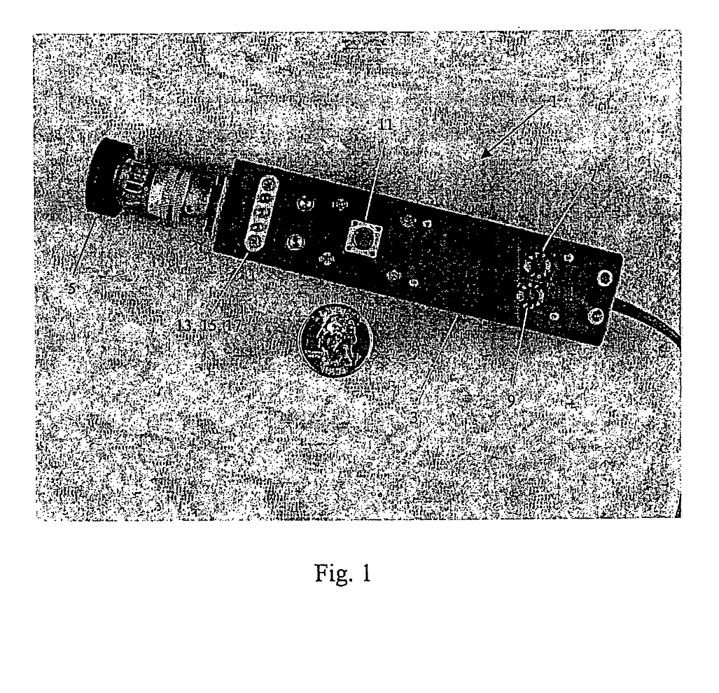

FIG. 1 illustrates an embodiment of a device 1 according to the present invention. The embodiment shown in FIG. 1 includes a hand-held member 3. A probe tip 5 is located at one end of the hand-held member. Any hand-held member according to the present invention typically has a size, shape, texture, contour and/or other design features that facilitate its convenient clinical use. A pair of rotary potentiometers 7 and 9 permit the target temperature of the probe tip to be adjusted. One rotary potentiometer adjusts the ten's unit of the temperature and the other rotary potentiometer adjusts the one's. A push button 11 permits the device to be turned on and off. This embodiment includes three sets of lights 13, 15, and 17 to indicate temperature as described above. The device includes a wired attachment at least to a power source.

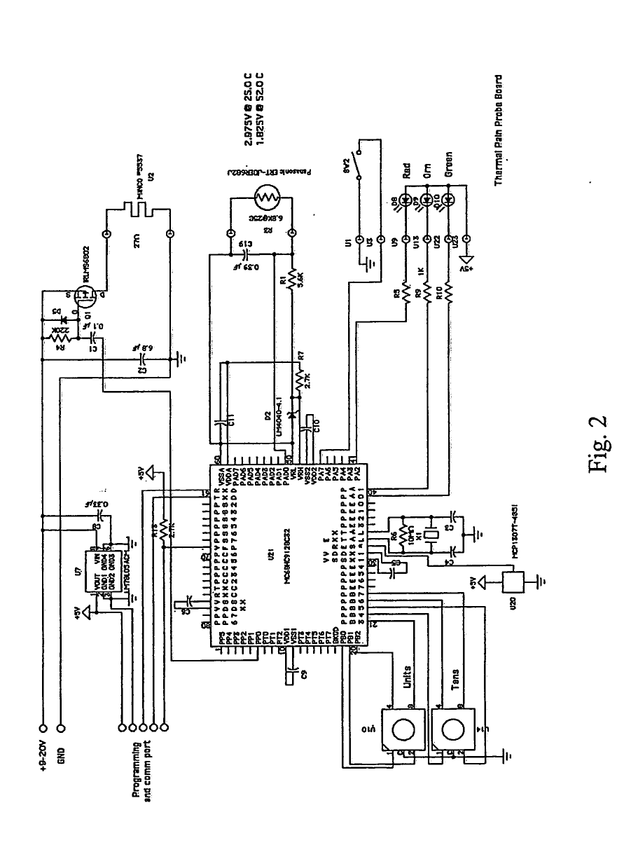

FIG. 2 illustrates an electrical schematic drawing for one embodiment of a device according to the present invention.

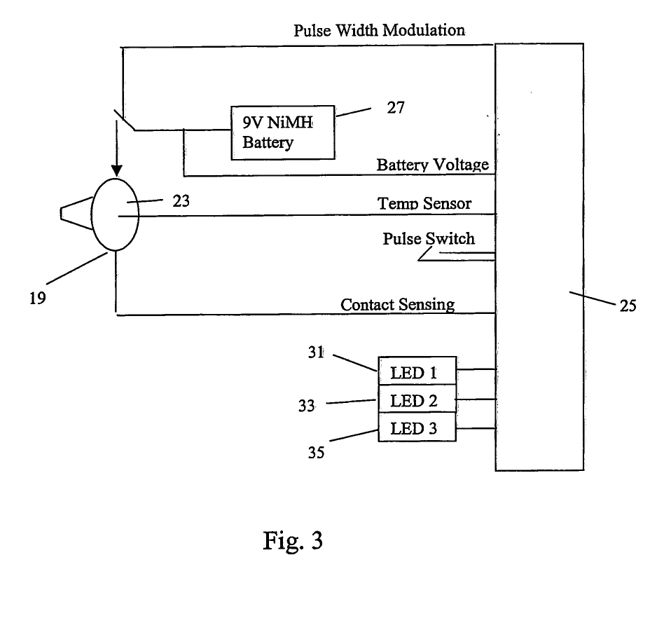

FIG. 3 schematically illustrates another embodiment of a device according to the present invention. The embodiment shown in FIG. 3 includes a probe tip 19 heated by a heater 21. A temperature sensor 23 is located on the probe tip. The device is connected to an analog/digital input/output card 25. The card 25 is operatively connected to the probe tip, the temperature sensor, and the heater. The card controls the heater with pulse width modulation. A battery 27 is also operatively connected to the heater and the card. The battery is a 9 volt nickel metal hydride battery. A pulse switch 29 can turn the device on and off. Three LEDs 31, 33, and 35 indicate the temperature as described above.

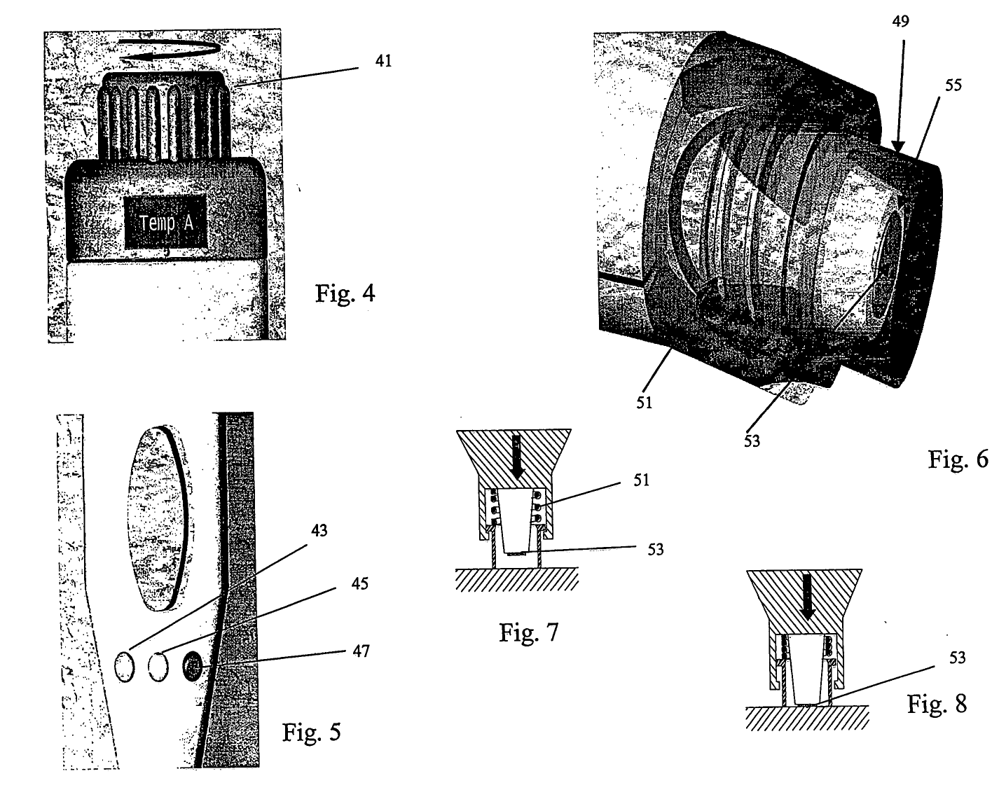

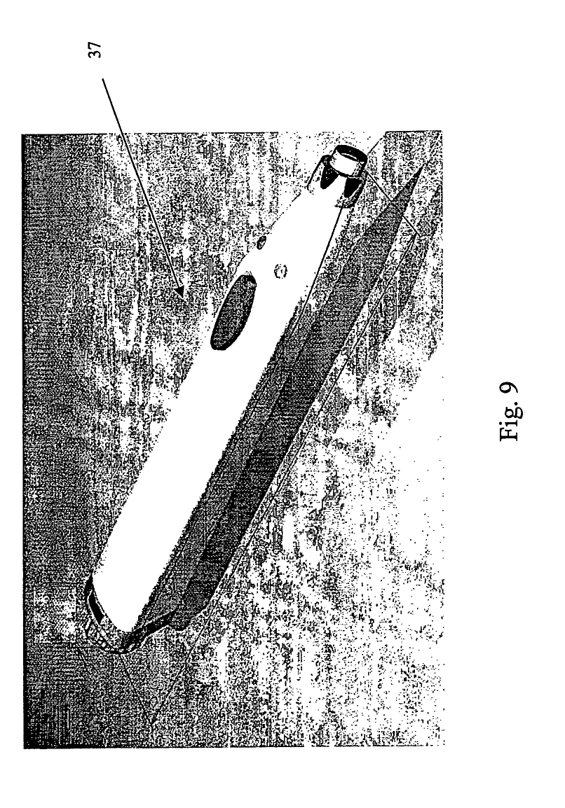

FIG. 9 illustrates another embodiment of a device 37 according to the present invention. Close-up views of portions of this embodiment are shown in FIGS. 4-8. This embodiment includes a power on-off button 39 illustrated in FIG. 5. Temperature of the probe tip may be selected with a rotary temperature control knob 41 located on an end of the housing opposite the probe tip. FIG. 4 illustrates a close-up view of the knob. This embodiment includes three colored LED indicators 43, 45, and 47, shown in close-up view in FIG. 5, to indicate device functioning as described above. The indicators can permit rapid assessment of when the tip has reached temperature after some change of set values. The process may be rapid, taking only a few hundred milliseconds.

The embodiment shown in FIG. 9 also includes a retractable shield in the form of a sleeve 49. The retractable sleeve includes an insulating housing. A tension spring 51 urges the retractable sleeve into an extended position. The spring is operatively connected to the indicators such that the red indicator, which indicates that the device is operational, will light when the sleeve is fully retracted with the spring compressed. The sleeve extends out from the housing beyond the probe tip 53.

This embodiment includes interchangeable probe heat tips with a skin contacting surface having a diameter of about 2 mm to about 8 mm. FIG. 6 illustrates a close-up partial cut-away view of the end of the housing that includes the probe and sleeve. FIGS. 7 and 8 represent cross-sectional views of the probe and sleeve. Force typically is required to push against the force of the spring and move the probe sleeve back until the tip makes contact with the test area of the patient's skin. Once the sleeve retracts and the probe tip makes full contact with the patient's skin, the test can proceed. The heat tip is arranged within an insulating housing 55.

The present invention also includes a method of testing patient response to excitation of neural fibers. In particular, the method can test thermal stimulation of small neural fibers. Heat is applied to a region of a patient's skin. The heat may be any non-injurious temperature. Typically, the temperature is about 32° C. to about 52° C. The surface area of the patient's skin to which the heat is applied may also vary. The region may be round and have a diameter of about 2 mm to about 8 mm. Of course, any shape and size region may be employed.

The patient response to the application of heat is noted. The size of the region that the heat is applied to, the temperature applied, the pattern of the region, the length of time that the heat is applied, as well as other parameters may be varied to determine patient response, patterns of nerve fibers, patient's threshold response to temperature, nerve fiber density, texture in nerve fiber alignment, loss of neural response as a function of time, differences in patient-to-patient response, and changes in time of any of these parameters. This list provides examples of parameters that may be noted and is not exhaustive. Other parameters may also be tested for.

To apply the heat, a region of a patient's skin is contacted a probe tip operatively connected to a heat source and operable to apply non-injurious heat to the region of the patient's skin. Heat may be applied to the probe tip with the heat source. A temperature of the probe tip may be sensed with a temperature sensor. The temperature of the probe tip may be controlled to maintain a target temperature. Patient response to the contact with the probe tip may be detected. At least one of the temperature of the probe tip, a size of the region of the patient's skin that the probe tip contacts, and a shape of the region of the patient's skin that the probe tip contacts may be varied.

To alter the size, shape, and pattern of the region to be contacted with elevated temperature, the probe tip may be changed to a probe tip having skin-contacting region with a different size, shape and/or pattern. The temperature response test data including at least one of test location on body, temperature threshold, contact area, and date may be recorded or stored in a memory structure.

The method may also include applying force to a retractable probe tip shield to permit the probe tip to contact the region of the patient's skin. Additionally, the method may include operatively connecting the probe tip, the temperature sensor and/or the heat source to a controller. Furthermore, the method may include operatively connecting the probe tip, the temperature sensor and/or the heat source to a power supply.

Several modes of patient testing may be utilized. One includes use of a single tip of selected area to map the threshold temperature response at multiple locations on the body by varying tip temperature above and below the nominal thermal pain threshold of about 45° C. By repeating testing using other tips of varied contact area, the effect of tip temperature and contact area may be separately determined. A second mode includes determining the variation in thermal pain threshold temperature with location on the body and produce maps of regions of neuropathy resulting either from loss of fiber density or loss of threshold pain response.

We claim:

1. A device operable to assess temperature response of small neural fibers, the device comprising:
a heat source;
a skin contacting probe tip comprising at least one skin contacting region operatively connected to the heat source and operable to apply a heat to regions of skin having varying surface areas;
a temperature sensor arranged in the vicinity of the probe tip and operable to detect a temperature of the probe tip; and
a controller operatively connected to the heat source and the temperature sensor to maintain a target temperature of the probe tip.

2. The device according to claim 1, wherein the probe tip includes a skin contacting surface having a varying skin contacting area.

3. The device according to claim 1, wherein the probe tip includes a skin contacting surface having a varying shape.

4. The device according to claim 1, wherein the skin contacting probe tip is interchangeable.

5. The device according to claim 4, wherein the interchangeable skin contacting probe tips have skin contacting surfaces including skin contacting areas having different areas.

6. The device according to claim 1, wherein the interchangeable skin contacting probes have different shapes of skin contacting surfaces.

7. The device according to claim 1, wherein the heat source is operable to generate a temperature of about 32° C. to about 53° C. in the probe tip.

8. The device according to claim 1, wherein the probe tip has a circular cross-section having a diameter of about 2 mm to about 12 mm.

9. The device according to claim 1, further comprising:
a retractable probe sleeve operable to shield at least a portion of the probe tip when the probe tip is not in use.

10. The device according to claim 9, wherein the probe tip is urged into contact with the skin against a extending force on the retractable shield.

11. The device according to claim 9, further comprising:
a spring operable to urge the retractable shield in an extended position.

12. The device according to claim 1, further comprising:
a power source operatively connectable to the heat source.

13. The device according to claim 1, wherein the heat source is a thin film heater.

14. The device according to claim 1, further comprising:
a memory operatively connected to the device and operable to record temperature response test data including at least one of test location on body, temperature threshold, contact area, and date.

15. The device according to claim 1, wherein the controller is arranged in a microcomputer operatively connected to the device.

16. The device according to claim 1, wherein the sensor comprises a surface mount thermistor.

17. The device according to claim 1, wherein the heat source comprises a source of optical energy.

18. The device according to claim 1, wherein the temperature sensor comprises at least one fluorescence producing element operative to fluoresce relative to the temperature of the probe tip.

19. The device according to claim 1, wherein response data is automatically collected.

20. A device operable to assess temperature response of small neural fibers, the device comprising:
a gripable housing;
a heat source arranged in the housing;
an interchangeable skin contacting probe tip extending from an end of the housing, the probe tip being operatively connected to the heat source and operable to apply non-injurious heat to regions of skin having varying surface areas;
a temperature sensor arranged in the vicinity of the probe tip and operable to detect temperature of the probe tip;
a controller operatively connected to the heat source and the temperature sensor to maintain a target temperature of the probe tip; and
a retractable probe tip shield extending around at least a portion of beyond the probe tip when the probe tip is not in use.

21. The device according to claim 20, wherein the controller is remote from the device.

22. The device according to claim 20, further comprising:
a memory operatively connected to the device and operable to record temperature response test data including at least one of test location on body, temperature threshold, contact area, and date.

23. The device according to claim 20, further comprising:
a power source arranged within the housing and operatively connected at least to the heat source.

24. The device according to claim 20, wherein the interchangeable probe tips include skin contract surfaces having different areas and/or different shapes.

25. The device according to claim 20, further comprising:
a visual indicator that the probe tip has reached the target temperature.

27. The device according to claim 20, wherein the heat source comprises a source of optical energy.

28. The device according to claim 20, wherein the temperature sensor comprises at least one fluorescence producing element operative to fluoresce relative to the temperature of the probe tip.

29. The device according to claim 20, wherein response data is automatically collected.

30. A method for testing patient response to excitation of neural fibers, the method comprising:
contacting a region of a patient's skin with a probe tip operatively connected to a heat source and operable to apply heat to the region of the patient's skin;
applying heat to the probe tip with the heat source;
sensing a temperature of the probe tip with a temperature sensor;
controlling the temperature of the probe tip to maintain a target temperature;
detecting patient response to the contact with the probe tip; and
varying at least one of the temperature of the probe tip, a size of the region of the patient's skin that the probe tip contacts, and a shape of the region of the patient's skin that the probe tip contacts.

31. The method according to claim 30, further comprising:
changing the probe tip to a probe tip having skin contacting region with a different size or shape.

32. The method according to claim 30, further comprising:
recording temperature response test data including at least one of test location on body, temperature threshold, contact area, and date.

33. The method according to claim 30, wherein the target temperature is about 32° C. to about 53° C.

34. The method according to claim 30, wherein the region of the patient's is round and has a diameter of about 2 mm to about 12 mm.

35. The method according to claim 30, further comprising:
applying force to a retractable probe tip shield to permit the probe tip to contact the region of the patient's skin.

36. The method according to claim 30, further comprising:
operatively connecting the probe tip to a controller.

37. The method according to claim 30, further comprising:
operatively connecting the heat source to a power supply.

38. The method according to claim 30, further comprising:
determining at least one of the patient's threshold response to temperature, nerve fiber density, texture in nerve fiber alignment, loss of neural response as a function of time and differences in patient-to-patient response.
